(19) United States
(12) Patent Application Publication
(10) Pub. No.: US 2007/0219506 A1
(43) Pub. Date: Sep. 20, 2007

(54) INJECTION SYRINGE

(76) Inventor: Gerhard Andersson, Boras (SE)

Correspondence Address: GAUTHIER & CONNORS, LLP, 225 FRANKLIN STREET, SUITE 2300, BOSTON, MA, 02110, US

(21) Appl. No.: 11/690,899

(22) Filed: Mar. 26, 2007

Related U.S. Application Data
(63) Continuation of application No. PCT/SE05/01443, filed on Sep. 29, 2005.

(30) Foreign Application Priority Data
Sep. 29, 2004 (SE) 0402361-0

(51) Int. Cl. A61M 5/00

(52) U.S. Cl. 604/207

(57) ABSTRACT

The present invention relates to a disassemble injection syringe, in particularly suited for diabetics for the administration of insulin, whereby an ampoule part containing a pre-filled amount of injection solution between an injection needle and a plunger, and a dosage of injection solution and mechanism bringing the plunger rod forward, are arranged releasably side-by-side parallel to each other in a first inactive position, and are arranged to be moved parallel axially relative each other adjustment into a coaxial position for an injection cooperation.

### TECHNICAL FIELD

The present invention relates to disassemble injection syringe in particular suited for diabetics for the injection of insulin

### BACKGROUND OF THE INVENTION

Injection administrations of different types are carried out using different injection syringes which generally encompass a cylindrical space which at one end thereof is provided with or can be provided with an injection needle suitable to the type of injection intended (e.g s.c i.v. i.m. i.p.) and in the other end thereof is provided a plunger with a plunger rod connected thereto

In most cases an injection solution is drawn up from a bottle/vial) into the cylindrical space prior to an injection or an ampoule is connected into a cylindrical space being a syringe body into which the plunger rod is arranged to operate A great number of different solutions of the design of injection syringes have thus been proposed during the years

U.S. Pat. No. 5,197,954 discloses an injection syringe in which the injection needle is present in a front part being provided with a hinge which can be pivoted up alongside the cylindrical space in order to thus reduce the risk of damage when one handles a used syringe

U.S. Pat. No. 4,011,868 discloses an injection syringe with plunger and plunger rod wherein the plunger rod can be bent backwards and be placed alongside the cylindrical space when the plunger is present in a rear position The bending/pivoting takes place along a bending line arranged in a separation of the plunger rod In the discløsure it is also described that the injection needle unit can be introduced and stored in the plunger rod The object of the invention is to make it possible having pre-filled syringes without risking unintended spraying of injection liquid

U.S. Pat. No. 4,221,218 discloses a syringe having an ampoule part of a piece of straight glass or tube of inert material a rubber plunger at the end of the ampoule part a plunger rod part being turnable 180° including a joint to be joined to the rear end of the ampoule receiving the plunger

GB-A-2,233,564 discloses a medical injection syringe having a plunger rod being turnable 180° as well in relation to a plunger provided ampoule part

WO97/29798 discloses an injection syringe having an ampoule whereby the syringe comprises an axially extending body having an axially extending hollow opening arranged to receive an ampoule part a connecting part at the distal end to receive an injection needle a plunger device to be brought into the proximal end whereby the plunger is attached to the proximal end of the syringe body to provide a pivotable joint wherein the plunger and the longitudinal axis of the plunger is turned from a first position 180° to a second position in relation to the ampoule part and the longitudinal axis of the ampoule to be positioned in a position alongside the ampoule part to facilitate the use of the plunger part to the plunger of the ampoule

These injection syringes thus include system to join the plunger rod to the ampoule part by means of a turning 180° over a hinge joint

To diabetics who have to inject themselves several times a day with a solution of insulin and thereby permanently have to bring with them a syringe or syringes there is today that which is called insulin pens i.e. an injection syringe of disposable type containing a pre-filled ampoule and a head with a dosage device for adjustment of the correct dosage which is determined by means of a blood glucose test done by the diabetic himself The dose is also related to an expected body activity and meals The insulin pens are rather long and fits poorly in a pocket Even jacket inside pockets may sometimes be too shallow The mode of today also often dictates slimmed clothes where it is difficult to hide or event be able to place an insulin pen

Many insulin pens are besides rather coarse i.e. have a significant diameter

### SUMMARY OF THE PRESENT INVENTION

The present invention relates to a disassemble injection syringe comprising an ampoule part and a plunger rod/dosage part which are placed in a sidewise relationship relative each other when the injection syringe is not in use ad which are operated to be brought together in a common axial relationship in relation to each other at use.

In particular the present invention is characterized in that an ampoule part containing a pre-filled amount of injection solution between an injection needle and a plunger and a dosage unit containing a plunger rod a device to adjust a dosage of injection solution and mechanism bringing the plunger rod forward whereby the ampoule part and the dosage part are arranged releasably side-by-side parallel to each other in a first inactive position and are arranged to be moved parallel axially relative each other adjustment into a coaxial position for an injection cooperation

A preferred embodiment of the invention is characterized in that either of the ampoule part or the dosage part

A preferred embodiment of the invention is characterized in that either of the ampoule part and the dosage part comprises a groove for receiving a protrusion running in parallel with the longitudinal axis of the ampoule part or the dosage part respectively which protrusion is arranged dosage part or the ampoule part respectively whereby the protrusion is axially displaceable and rotatable in said groove in order to bring the parts into a coaxial position in relation to each other

A preferred embodiment of the invention is characterized in that the ampoule part and the dosage part comprise a pivotable lever system connecting the parts whereby the ampoule part is axially displaceable and rotatable in relation to said dosage part to bring the parts into a coaxial position relative each other whereby the longitudinal axis of the ampoule part and the longitudinal axis of the dosage part respectively are arranged to be displaced parallel axially relative each other by means of the lever system to adjustment into a coaxial position for injection cooperation

Another preferred embodiment of the invention is characterized in that the syringe is intended for single use.

A further preferred embodiment of the invention is characterized in that the syringe is arranged to receive an exchangeable ampoule containing an injection solution

Another further preferred embodiment of the invention is characterized in that the syringe is particularly suited for diabetics for the injection of insulin

### DETAILED DESCRIPTION OF THE PRESENT INVENTION

The present invention will be described more in detail in the following with reference to the accompanying drawing wherein

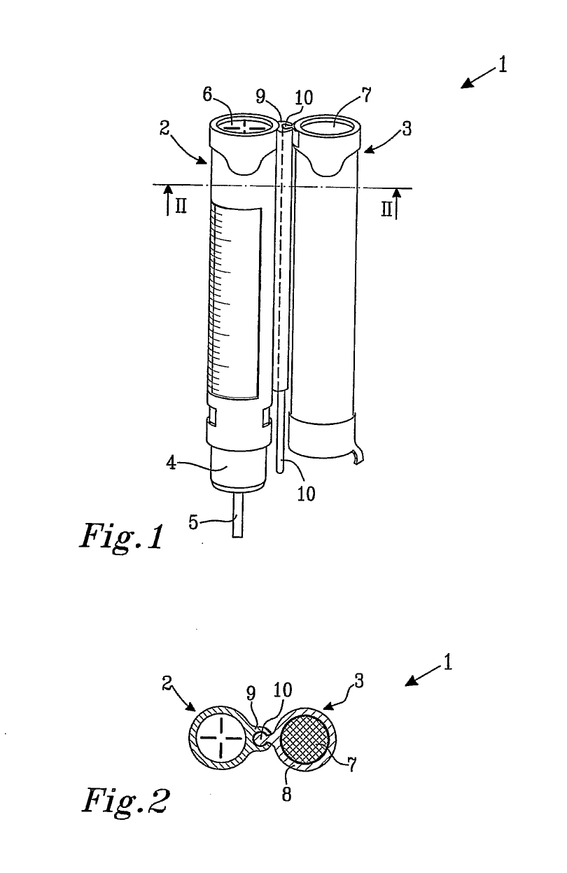

FIG. 1 shows a first embodiment of an injection syringe according to the present invention arranged in a sidewise relationship

FIG. 2 shows the injection syringe according to FIG. 1 in a cross-section along the line II-II

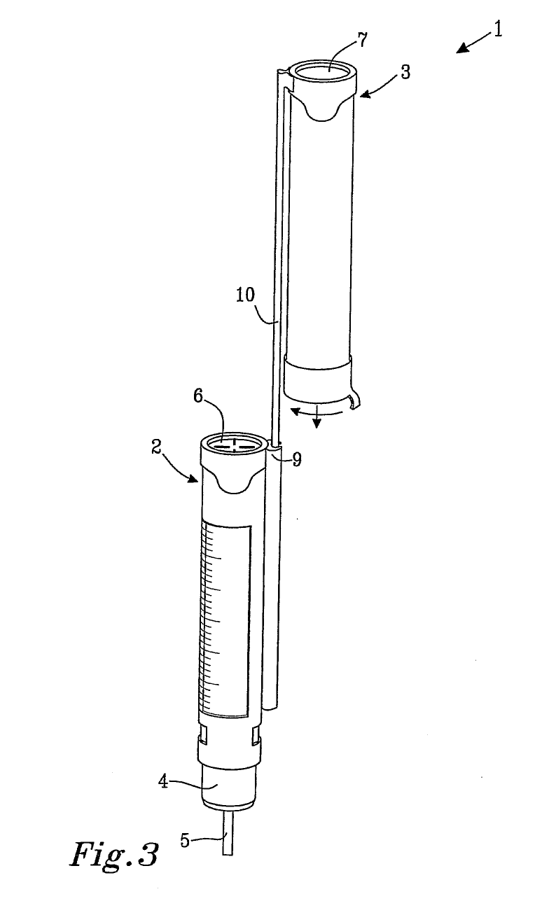

FIG. 3 shows the injection syringe according to FIG. 1 during joining to one unit

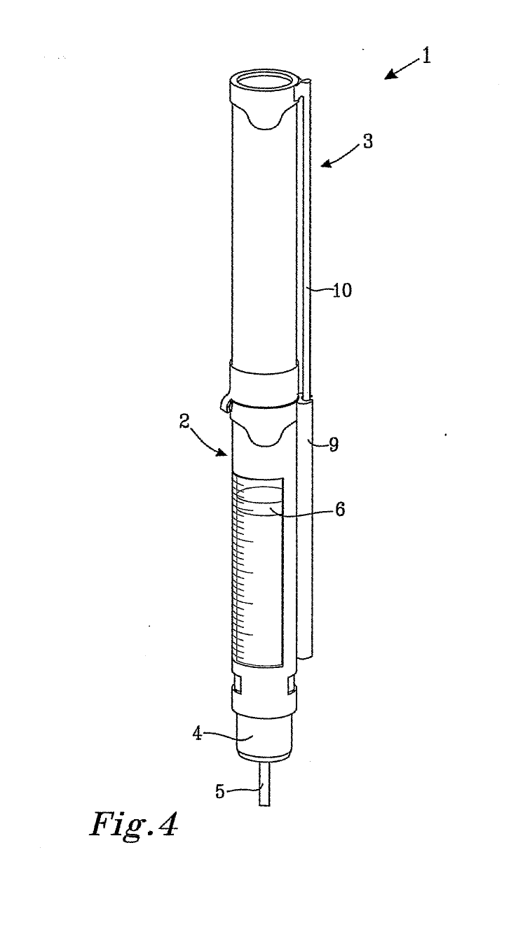

FIG. 4 shows the injection syringe according to FIG. 1 in a joined condition

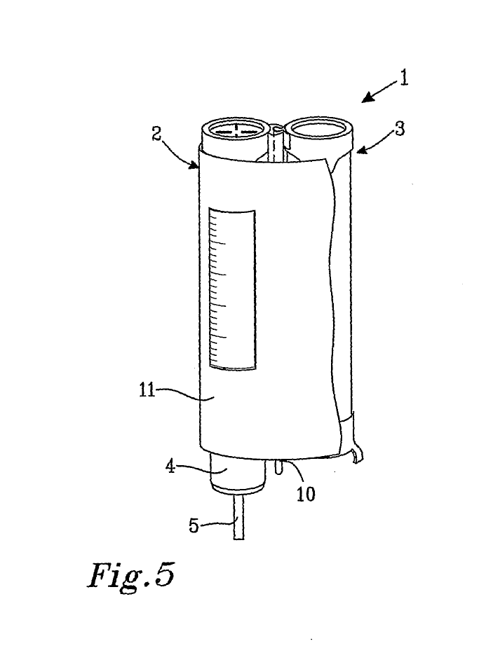

FIG. 5 shows an injection syringe according to FIG. 1 having an outer envelope

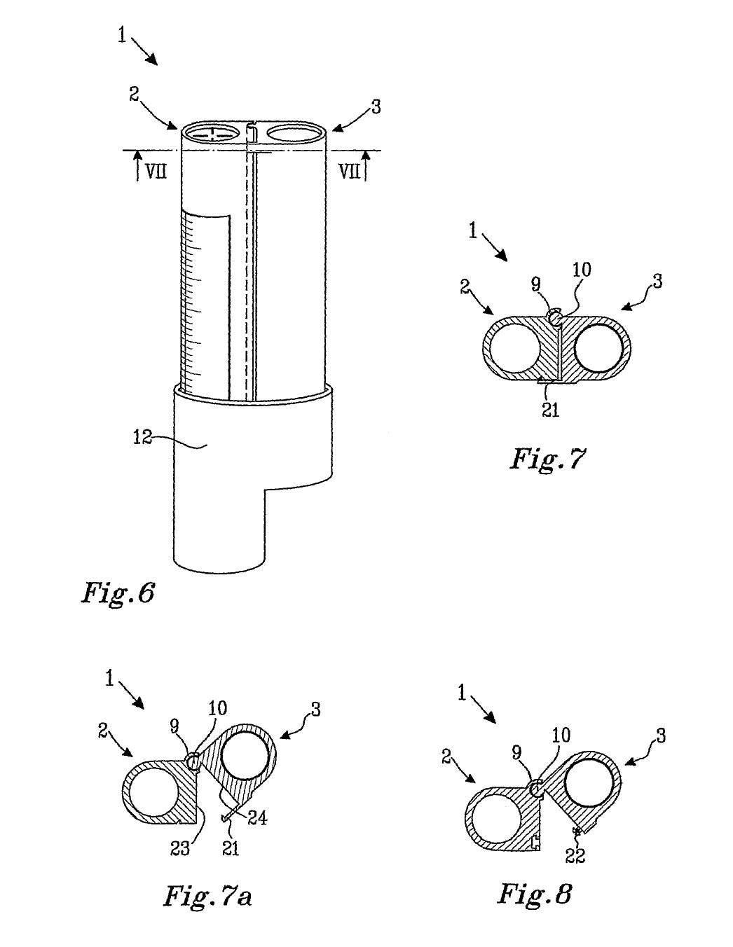

FIG. 6 shows a second embodiment of an injection syringe of the present invention arranged in a sidewise relationship

FIG. 7 shows the injection syringe according to FIG. 6 in a cross-section along the line VII-VII

FIG. 7a shows the injection syringe according to FIG. 7 in a cross-section along line VII-VII in a partly open condition

FIG. 8 shows a variety of the injection syringe according to FIG. 6 in a cross-section along the line VII-VII

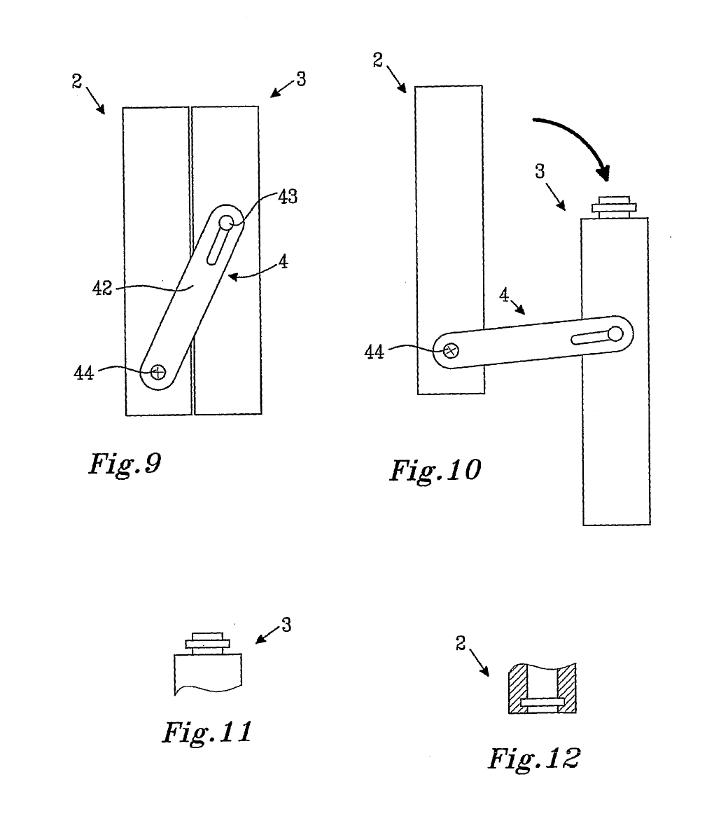

FIG. 10 shows a third embodiment of an injection syringe according to the present invention arranged in a sidewise relationship

FIGS. 11 and 14 show the injection syringe according to FIG. 10 during joining to one unit

FIG. 12 shows one part of the injection syringe according to FIG. 10 to provide for a joined condition

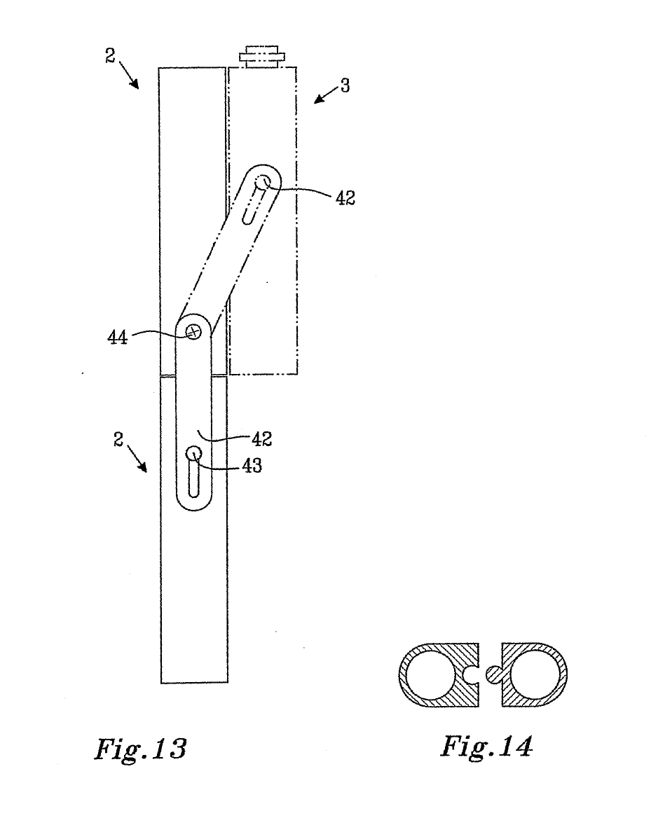

FIG. 13 shows a second part of the injection syringe according to FIG. 10 to provide for a joined condition

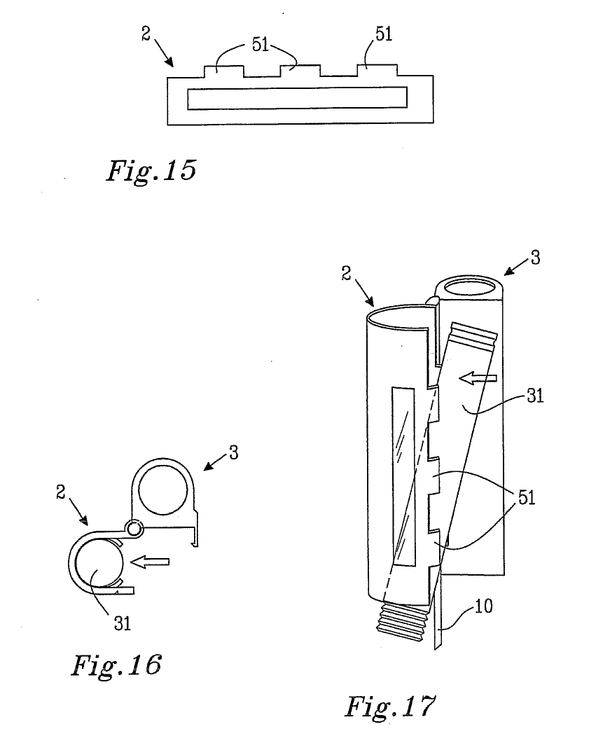

FIG. 15 shows a detail of an injection syringe according to a further embodiment in a cross-section

FIG. 16 shows a detail of an injection syringe according to a further embodiment in a cross-section

1 denotes generally a disassemble injection syringe according to the present invention The injection syringe comprises an ampoule part 2 and a plunger rod/dosage part 3.

The ampoule part 2 is generally provided with at its front end 4 an injection needle (in the picture the needle is protected by a needle protecting shield 5) of such a type being suited for the injection to be carried out i.e. quite often a thin cannula used for subcutaneous injections och coarser cannulas for e.g. intra muscular injections The ampoule part 2 is completely or partly transparent so that its content can be controlled visually In the rear part of the ampoule 2 a plunger 6 is arranged which is arranged to receive or in any other way cooperate with a plunger rod 7 which will be described in closer detail below

The plunger rod/dosage part 3 comprises a housing 8 which encloses a plunger rod 7 which is fed/moved forward via a thread system (not shown) The thread system comprises an adjustment mechanism which means that a certain forward movement can be carried out after adjustment in order to administer a certain predetermined dose when the plunger rod 7 is being moved forward The thread mechanism contains such a function as well that the plunger rod can be retracted to a zero-position in order to be able to be brought forward to a prior final point of injection Finally the thread mechanism comprises an outer injection trigger which at an axial pressure activates the plunger rod/plunger to be fed a determined length

The ampoule part 2 has along its side a peripherally axially to the longitudinal axis of the ampoule extending groove 9. On the side of the plunger rod/dosage unit 3 a peripherally extending protrusion 10, corresponding to the groove 9 is running The groove 9 is arranged to receive the protrusion 10 and is arranged in such a way that the protrusion 10 using simple hand power can be moved in the groove 9 (FIG. 3) whereupon the plunger rod/dosage unit 3 is turned around the protrusion 10 in the groove 9 in a plane perpendicular to the longitudinal axis of the plunger rod part such that this will axially coincide with the ampoule part 2 whereupon the parts 2 and 3 are removably attached to each other Hereby the protrusion 10 has a length exceeding the length of the groove 9 such that the protrusion remains in the groove 9 during the turning operation and will thereby become a guide to the turning over Then a predetermined injection dosage is adjusted whereby the plunger rod 7 is introduced into the ampoule part 2 and is connected to the plunger 6, the injection trigger is actuated and injection is made in an intended way When the injection is finished the plunger rod is returned the plunger rod/dosage part 3 is released and is turned back to a sidewise position and is moved down along the ampoule part 2 to form a compact unit The unit can be received in an envelope 11 if so desired The envelope 11 snaps around the injection syringe in a compressed compact condition so that the plunger rod/dosage part 3 can be moved up and be placed over the ampoule part 2.

FIG. 6 shows a second embodiment of the invention where the groove 9 and the protrusion 10 respectively are placed in the respective corner of the housings of the ampoule part 2 and the plunger rod/dosage part 3 respectively In the opposite corner of the plunger rod part 3 a hook 21 is arranged which is either an outer hook 21 or an inner catch 22 locking the parts to each other in a compact position Hereby the respective parts have opposing plane surfaces 23 and 24 respectively In the embodiment shown there is a part envelope 12 receiving the lower needle provided end 4 of the ampoule part 2 and an upper part of the upper part of the plunger rod/dosage part 3 in a compact position

According to a variety of this embodiment (FIG. 16) an exchangeable ampoule 31 can be used whereby the ampoule 31 is either loaded from above i.e. axially into the ampoule part: or is introduced sidewise radially into the ampoule part 2. In the later case the whole side of the ampoule 2 can be a snap holder to retain the ampoule 31 or may the edge of the sidewise opened ampoule part 2 be provided with tongues/protrusions 51 forming snaps to retain the ampoule 31

FIG. 10 shows a further embodiment of the invention where the ampoule part and the dosage part are joined using a lever system 41 Hereby a lever arm 42 is rotatably arranged between the two parts and is arranged to the respective part via a rotation axis 43 and 44 on the respective part 2, 3. In a non-use position the parts 2 3 are placed alongside each other and at use the lever arm 42 and the ampoule part 2 are turned outwards so that the ampoule part 2 will be placed coaxially to the dosage part 3. The lever arm has in connection with each rotation axis a cross section making them abutting the respective part to lock the same while at the middle thereof it has a cross-section which makes it locks the parts when the coincide axially This means that one has a double snapping function one in an inactive position and one in an active position Depending on the geometrical design of the lever system the ampoule part 2 can be introduced along an arrow towards the ampoule part or be turned simultaneously 18Oo (FIG. 11) to arrive towards the plunger rod part 3.

FIG. 15 shows a cross-section of an embodiment wherein the groove 9 is a more integrated part of e.g. the ampoule wall and wherein the ampoule wall and the wall of the plunger rod run against each other over a considerable surface area This embodiment forms a well coherent shape having a substantially integrated outer contour

1. A disassemble injection syringe characterized in an ampoule part containing a pre-filled amount of injection solution between an injection needle and a plunger and a dosage unit containing a plunger rod a device to adjust a dosage of injection solution and mechanism bringing the plunger rod forward whereby the ampoule part and the dosage part are arranged releasably side-by-side parallel to each other in a first inactive position and are arranged to be moved parallel axially relative each other adjustment into a coaxial position for an injection cooperation.

2. An injection syringe according to claim 1, wherein either of the ampoule part or the dosage part comprises a groove for receiving a protrusion running in parallel with the longitudinal axis of the ampoule part or the dosage part respectively which protrusion is arranged to the dosage part or the ampoule part respectively whereby the protrusion is axially displaceable.

3. An injection syringe according to claim 1 wherein either of the ampoule part and the dosage part comprises a groove for receiving a protrusion running in parallel with the longitudinal axis of the ampoule part or the dosage part respectively which protrusion is arranged to the dosage part or the ampoule part respectively whereby the protrusion is axially displaceable and rotatable in the groove in order to bring the parts into a coaxial position in relation to each other.

4. An injection syringe according to claim 1 wherein the ampoule part and the dosage part comprise a pivotable lever system connecting the parts whereby the ampoule part is axially displaceable and rotatable in relation to said dosage part to bring the parts into a coaxial position relative each other whereby the longitudinal axis of the ampoule part and the longitudinal axis of the dosage part respectively are arranged to be displaced parallel axially relative each other by means of the lever system to adjustment into a coaxial position for injection cooperation.

5. An injection syringe according to claim 1 wherein the syringe is intended for single use.

6. An injection syringe according to claim 1 wherein the syringe is arranged to receive an exchangeable ampoule containing an injection solution.

7. An injection syringe according to claim 1 wherein, the syringe is particularly suited for diabetics for the injection of insulin.

8. An injection syringe according to claim 1 wherein the ampoule part and the dosage part are contained on a connecting envelope.
